SEQUENCE LISTING (67 sequences)

SEQ ID NO: 1 - DNA, 21 bp, synthetic construct
  misc_feature 1..21: note = MS2
  source 1..21: mol_type = other DNA
aacatgaggatcacccatgtc

SEQ ID NO: 2 - DNA, 19 bp, synthetic construct
  misc_feature 1..19: note = Lambda Nut-L
  source 1..19: mol_type = other DNA
gggccctgaagaagggccc

SEQ ID NO: 3 - DNA, 15 bp, synthetic construct
  misc_feature 1..15: note = Lambda Nut-R
  source 1..15: mol_type = other DNA
gccctgaaaaagggc

SEQ ID NO: 4 - DNA, 21 bp, synthetic construct
  misc_feature 1..21: note = Qb
  source 1..21: mol_type = other DNA
aatgcatgtctaagacagcat

SEQ ID NO: 5 - DNA, 28 bp, synthetic construct
  misc_feature 1..28: note = BIV TAR
  source 1..28: mol_type = other DNA
ggctcgtgtagctcattagctccgagcc

SEQ ID NO: 6 - DNA, 30 bp, synthetic construct
  misc_feature 1..30: note = STNV
  source 1..30: mol_type = other DNA
ccttttcaagacatgcaacaatgcacacag

SEQ ID NO: 7 - DNA, 25 bp, synthetic construct
  misc_feature 1..25: note = PP7
  source 1..25: mol_type = other DNA
ggagcagacgatatggcgtcgctcc

SEQ ID NO: 8 - DNA, 20 bp, Homo sapiens
  source 1..20: mol_type = genomic DNA
ggacaggtcctcctgggagc

SEQ ID NO: 9 - DNA, 20 bp, Homo sapiens
  source 1..20: mol_type = genomic DNA
tcctgtccatctccaccatc

SEQ ID NO: 10 - DNA, 20 bp, Homo sapiens
  source 1..20: mol_type = genomic DNA
tctaggaggagtcacaaaaa

SEQ ID NO: 11 - DNA, 20 bp, Homo sapiens
  source 1..20: mol_type = genomic DNA
cgctatatgttaggattcga

SEQ ID NO: 12 - DNA, 20 bp, Homo sapiens
  source 1..20: mol_type = genomic DNA
ggcgaggggcaacttccatc

SEQ ID NO: 13 - DNA, 20 bp, Homo sapiens
  source 1..20: mol_type = genomic DNA
cacactatggcgcacgtccg

SEQ ID NO: 14 - DNA, 20 bp, Homo sapiens
  source 1..20: mol_type = genomic DNA
tatctacccacccgtcccca

SEQ ID NO: 15 - DNA, 20 bp, Homo sapiens
  source 1..20: mol_type = genomic DNA
aacattaacccagaggggtc

SEQ ID NO: 16 - DNA, 20 bp, Homo sapiens
  source 1..20: mol_type = genomic DNA
acatgaagaaatcagcggag

SEQ ID NO: 17 - DNA, 20 bp, Homo sapiens
  source 1..20: mol_type = genomic DNA
accccagctgtgctgcagag

SEQ ID NO: 18 - DNA, 20 bp, Homo sapiens
  source 1..20: mol_type = genomic DNA
gctcagtggctgggcagcag

SEQ ID NO: 19 - DNA, 20 bp, Homo sapiens
  source 1..20: mol_type = genomic DNA
tgcatggccactggccggcc

SEQ ID NO: 20 - DNA, 20 bp, Homo sapiens
  source 1..20: mol_type = genomic DNA
aactttgcccgtcagtccca

SEQ ID NO: 21 - DNA, 20 bp, Homo sapiens
  source 1..20: mol_type = genomic DNA
ttgccagcgtgcaggtgtta

SEQ ID NO: 22 - DNA, 20 bp, Homo sapiens
  source 1..20: mol_type = genomic DNA
aaatttcacctctgatttcg

SEQ ID NO: 23 - DNA, 20 bp, Homo sapiens
  source 1..20: mol_type = genomic DNA
cagactcaaatcagccacag

SEQ ID NO: 24 - DNA, 20 bp, Homo sapiens
  source 1..20: mol_type = genomic DNA
cttgggacaagtgaagagaa

SEQ ID NO: 25 - DNA, 20 bp, Homo sapiens
  source 1..20: mol_type = genomic DNA
ctctctgacaaagatacggt

SEQ ID NO: 26 - DNA, 20 bp, Homo sapiens
  source 1..20: mol_type = genomic DNA
tcatcaggatctagtgttag

SEQ ID NO: 27 - DNA, 20 bp, Homo sapiens
  source 1..20: mol_type = genomic DNA
aatagtccaaagacccattg

SEQ ID NO: 28 - DNA, 20 bp, Homo sapiens
  source 1..20: mol_type = genomic DNA
ctttcacaatctgctagcaa

SEQ ID NO: 29 - DNA, 20 bp, Homo sapiens
  source 1..20: mol_type = genomic DNA
aggcaggggagtgaccacgc

SEQ ID NO: 30 - DNA, 20 bp, Homo sapiens
  source 1..20: mol_type = genomic DNA
gatgaggatgaggttctgcg

SEQ ID NO: 31 - DNA, 20 bp, Homo sapiens
  source 1..20: mol_type = genomic DNA
tctggccgcccagctctgcc

SEQ ID NO: 32 - DNA, 20 bp, Homo sapiens
  source 1..20: mol_type = genomic DNA
gtggtctcatgtcacggggg

SEQ ID NO: 33 - DNA, 20 bp, Homo sapiens
  source 1..20: mol_type = genomic DNA
aggtggtctcatgtcacggg

SEQ ID NO: 34 - DNA, 20 bp, Homo sapiens
  source 1..20: mol_type = genomic DNA
aggccgctggcagtgagcgt

SEQ ID NO: 35 - DNA, 20 bp, Homo sapiens
  source 1..20: mol_type = genomic DNA
acagtactcggccacaccat

SEQ ID NO: 36 - DNA, 20 bp, Homo sapiens
  source 1..20: mol_type = genomic DNA
agcaggtctcggctccaatc

SEQ ID NO: 37 - DNA, 20 bp, Homo sapiens
  source 1..20: mol_type = genomic DNA
aggtctcggctccaatcagg

SEQ ID NO: 38 - DNA, 20 bp, Homo sapiens
  source 1..20: mol_type = genomic DNA
cgctgggaacacatccactc

SEQ ID NO: 39 - DNA, 20 bp, Homo sapiens
  source 1..20: mol_type = genomic DNA
ggaggaacaaaggacgtaga

SEQ ID NO: 40 - DNA, 20 bp, Homo sapiens
  source 1..20: mol_type = genomic DNA
tcggaagcaaacaagctgag

SEQ ID NO: 41 - DNA, 20 bp, Homo sapiens
  source 1..20: mol_type = genomic DNA
ttctgggataccaacagcat

SEQ ID NO: 42 - DNA, 20 bp, Homo sapiens
  source 1..20: mol_type = genomic DNA
caagaggtttggttaatcat

SEQ ID NO: 43 - DNA, 20 bp, Homo sapiens
  source 1..20: mol_type = genomic DNA
tttcagctggagtgctcctc

SEQ ID NO: 44 - DNA, 20 bp, Homo sapiens
  source 1..20: mol_type = genomic DNA
cttccgataagctgttgcaa

SEQ ID NO: 45 - DNA, 20 bp, Homo sapiens
  source 1..20: mol_type = genomic DNA
gagatctcctaattgttgac

SEQ ID NO: 46 - DNA, 20 bp, Homo sapiens
  source 1..20: mol_type = genomic DNA
tgattctccagtcaacaatt

SEQ ID NO: 47 - DNA, 20 bp, Homo sapiens
  source 1..20: mol_type = genomic DNA
ggagggttgactgtctacac

SEQ ID NO: 48 - DNA, 20 bp, Homo sapiens
  source 1..20: mol_type = genomic DNA
aagatacacatctctctttg

SEQ ID NO: 49 - DNA, 20 bp, Homo sapiens
  source 1..20: mol_type = genomic DNA
gtaaatttgacctactcaca

SEQ ID NO: 50 - DNA, 20 bp, Homo sapiens
  source 1..20: mol_type = genomic DNA
ttagtggttgccttgtgagt

SEQ ID NO: 51 - DNA, 20 bp, Homo sapiens
  source 1..20: mol_type = genomic DNA
tctgaagtcattcctagcag

SEQ ID NO: 52 - DNA, 20 bp, Homo sapiens
  source 1..20: mol_type = genomic DNA
agcaaagcttatttacttgt

SEQ ID NO: 53 - DNA, 20 bp, Homo sapiens
  source 1..20: mol_type = genomic DNA
cagaggactcagatatatat

SEQ ID NO: 54 - DNA, 20 bp, Homo sapiens
  source 1..20: mol_type = genomic DNA
tcagttaagtctacatgaaa

SEQ ID NO: 55 - DNA, 20 bp, Homo sapiens
  source 1..20: mol_type = genomic DNA
ggacaaaatgatgggaagtt

SEQ ID NO: 56 - DNA, 20 bp, Homo sapiens
  source 1..20: mol_type = genomic DNA
aagcattatccattgctgtg

SEQ ID NO: 57 - DNA, 20 bp, Homo sapiens
  source 1..20: mol_type = genomic DNA
ctcctttttggcccagctca

SEQ ID NO: 58 - DNA, 20 bp, Homo sapiens
  source 1..20: mol_type = genomic DNA
tgatccaaaattatctgcaa

SEQ ID NO: 59 - DNA, 20 bp, Homo sapiens
  source 1..20: mol_type = genomic DNA
ggcccccaacctatactgtc

SEQ ID NO: 60 - DNA, 20 bp, Homo sapiens
  source 1..20: mol_type = genomic DNA
aatggctggagctgatcacg

SEQ ID NO: 61 - DNA, 20 bp, Homo sapiens
  source 1..20: mol_type = genomic DNA
gggaacctgacagtataggt

SEQ ID NO: 62 - DNA, 20 bp, Homo sapiens
  source 1..20: mol_type = genomic DNA
acggggcctccttaccagaa

SEQ ID NO: 63 - DNA, 20 bp, Homo sapiens
  source 1..20: mol_type = genomic DNA
cactacaaggctcggagctc

SEQ ID NO: 64 - DNA, 20 bp, Homo sapiens
  source 1..20: mol_type = genomic DNA
gcagccgctcgctgcagcag

SEQ ID NO: 65 - DNA, 20 bp, Homo sapiens
  source 1..20: mol_type = genomic DNA
gggcccctgcggccaccccg

SEQ ID NO: 66 - DNA, 20 bp, Mus musculus
  source 1..20: mol_type = genomic DNA
aagggtagcctcccctagaa

SEQ ID NO: 67 - DNA, 20 bp, Mus musculus
  source 1..20: mol_type = genomic DNA
ggcccagggctccaatcaga

(19) United States
(12) Patent Application Publication
(10) Pub. No.: US 2025/0121041 A1
(43) Pub. Date: Apr. 17, 2025

(54) HEMOSTATIC COMPOSITION CONTAINING RECOMBINANT HUMAN CLOTTING FACTORS, AND METHOD OF PRODUCING

(71) Applicants: THE GOVERNMENT OF THE UNITED STATES, AS REPRESENTED BY THE SECRETARY OF THE ARMY, Fort Detrick, MD (US); REGENTS OF THE UNIVERSITY OF MINNESOTA, Minneapolis, MN (US)

(72) Inventors: Daniel Taylor LAMMERS, Fircrest, WA (US); Matthew Joseph ECKERT, Tacoma, WA (US); Mark John OSBORN, Minneapolis, MN (US); Jason Ray BINGHAM, Tacoma, WA (US); Colby J. FESER, St. Paul, MN (US)

(21) Appl. No.: 18/726,717

(22) PCT Filed: Jan. 6, 2023
(86) PCT No.: PCT/US2023/060222

§ 371 Date: Jul. 3, 2024

Related U.S. Application Data
(60) Provisional application No. 63/266,532, filed on Jan. 7, 2022.

(51) Int. Cl. A61K 38/48; A61K 38/36; C07K 14/75; C12N 5/071; C12N 9/22; C12N 9/64; C12N 9/74; C12N 15/11; C12N 15/90; C12P 21/00

(52) U.S. Cl. CPC A61K 38/4846; A61K 38/363; A61K 38/4833; C07K 14/75; C12N 5/067; C12N 9/22; C12N 9/6429; C12N 9/6432; C12N 9/6437; C12N 9/644; C12N 15/11; C12N 15/907; C12P 21/00; C12Y 304/21005; C12Y 304/21006; C12Y 304/21021; C12Y 304/21022; C12N 2310/20

(57) ABSTRACT

This invention relates to a wholly recombinant four factor hemostatic complex concentrate (4F-PCC) composition, human liver cell-produced product. Methods of producing a hemostatic composition using a hepatocyte cell line and a CRISPR/Cas9 gene activation multiplexing, which allows for simultaneous and high level expression of the FII, FVII, FIX, and FX clotting factors. The resultant product is a recombinant resuscitation solution to aid in mitigation of coagulopathy, reversal of coagulopathic states, and fluid resuscitation.

### CROSS REFERENCE TO RELATED APPLICATIONS

This patent application is a national stage patent application of International Patent Application No. PCT/US2023/060222, filed Jan. 6, 2023, that claims priority to U.S. Provisional Patent Application No. 63/266,532, filed on Jan. 7, 2022, the contents of each of which are hereby incorporated by reference in their entirety.

### STATEMENT AS TO RIGHTS OR INVENTIONS MADE UNDER SPONSORED RESEARCH AND DEVELOPMENT

This invention was made with government support under W81XWH-20-C-0052 awarded by the Medical Research and Development Command. The government has certain rights in the invention.

### REFERENCE TO SEQUENCE LISTING SUBMITTED AS A COMPLIANT XML FILE (.XML)

Pursuant to the EFS-Web legal framework and 37 CFR §§ 1.821-825 (see MPEP § 2442.03(a)), a Sequence Listing in the form of a compliant XML file (entitled “3000050-016001_Sequence_Listing_ST26.xml” created on Jul. 3, 2024, and 62,479 bytes in size) is submitted concurrently with the instant application, and the entire contents of the Sequence Listing are incorporated herein by reference.

### BACKGROUND

Blood products are required to manage coagulopathy resulting from inherited bleeding disorders, trauma, surgery, and for managing coagulation therapy. These products include fresh frozen plasma (FFP), Cryoprecipitate (CRYO), Fibrinogen Concentrate (FC), and Prothrombin Complex Concentrate (PCC), and each are used to achieve/maintain hemostasis. Each of these agents are obtained from plasma that is pooled from multiple unrelated donors making them subject to supply chain shortages and costly pathogen screening. Concentrated products like FC and PCC represent more uniform products with minimized risk from pathogen contamination; however, the exorbitant cost ($5,000 per dose of PCC) is prohibitive. The lack of a stable, affordable, and safe source of coagulation therapy products represents a therapeutic and engineering gap.

### SUMMARY OF INVENTION

This invention describes a method to produce wholly recombinant hemostatic composition comprising four human clotting factors-FII, FVII, FIX, and FX. The composition is preferably a four factor prothrombin complex concentrate (4F-PCC) human liver cell produced product. Because hepatocytes are responsible for the synthesis of most coagulation factors, the inventors employed a hepatocyte cell line to demonstrate the central hypothesis that CRISPR/Cas9 gene activation multiplexing allows for simultaneous and high level expression of the 4-FPCC constituents: FII, FVII, FIX, and FX clotting factors. The resultant product is a recombinant resuscitation solution to aid in mitigation of coagulopathy, reversal of coagulopathic states, and fluid resuscitation.

In an embodiment, a method for producing recombinant human clotting factors FII, FVII, FIX and FX can comprise the steps of: in a human hepatocyte cell, expressing DNA for each of human clotting factors FII, FVII, FIX and FX and encoding the respective gene products in an engineered, non-naturally occurring Clustered Regularly Interspaced Short Palindromic Repeats (CRISPR)-Synergistic Activation Module (SAM) system, comprising one or more vectors comprising a first regulatory element operable in the human hepatocyte cell operably linked to a nucleotide sequence encoding a CRISPR-Cas system guide RNA that hybridizes with human clotting factor FII DNA sequence; a nucleotide sequence encoding a CRISPR-Cas system guide RNA that hybridizes with human clotting factor FVII DNA sequence; a nucleotide sequence encoding a CRISPR-Cas system guide RNA that hybridizes with human clotting factor FIX DNA sequence; and a nucleotide sequence encoding a CRISPR-Cas system guide RNA that hybridizes with human clotting factor FX DNA sequence; and a second regulatory element operable in the human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, transcriptional activation domains, optionally comprising transcriptional activation subunits, and aptamers capable to form RNA aptamer stem loops into the respective guide RNAs and which specifically bind/hybridize to the respective human clotting factor, whereby each guide RNA targets and hybridizes to the appropriate human clotting factor FII, FVII, FIX or FX sequence, whereby expression of gene products for human clotting factor FII, FVII, FIX and FX is altered; and, wherein the Cas9 protein and the guide RNAs do not naturally occur together This is referred to herein as the “multiplex” approach, system or method.

In an embodiment, a method for producing a composition comprising recombinant human clotting factors FII, FVII, FIX and FX, can comprise the steps of: in a first human hepatocyte cell, expressing DNA for human clotting factor FII and encoding human clotting factor FII gene product in an engineered, non-naturally occurring Clustered Regularly Interspaced Short Palindromic Repeats (CRISPR)-Synergistic Activation Module (SAM) system, comprising one or more vectors comprising a first regulatory element operable in the first human hepatocyte cell operably linked to a nucleotide sequence encoding a CRISPR-Cas system guide RNA that hybridizes with human clotting factor FII DNA sequence; a second regulatory element operable in the first human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA, whereby the guide RNA targets and hybridizes to the human clotting factor FIX sequence, whereby expression of gene product for human clotting factor FIX is altered; in a second human hepatocyte cell, expressing DNA for human clotting factor FVII and encoding human clotting factor FVII gene product in an engineered, non-naturally occurring Clustered Regularly Interspaced Short Palindromic Repeats (CRISPR)-Synergistic Activation Module (SAM) system, comprising one or more vectors comprising a third regulatory element operable in the second human hepatocyte cell operably linked to a nucleotide sequence encoding a CRISPR-Cas system guide RNA that hybridizes with human clotting factor FVII DNA sequence; a fourth regulatory element operable in the second human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA, whereby the guide RNA targets and hybridizes to the human clotting factor FVII sequence, whereby expression of gene product for human clotting factor FVII is altered; in a third human hepatocyte cell, expressing DNA for human clotting factor FIX and encoding human clotting factor FIX gene product in an engineered, non-naturally occurring Clustered Regularly Interspaced Short Palindromic Repeats (CRISPR)-Synergistic Activation Module (SAM) system, comprising one or more vectors comprising a fifth regulatory element operable in the third human hepatocyte cell operably linked to a nucleotide sequence encoding a CRISPR-Cas system guide RNA that hybridizes with human clotting factor FIX DNA sequence; a sixth regulatory element operable in the third human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA, whereby the guide RNA targets and hybridizes to the human clotting factor FIX sequence, whereby expression of gene product for human clotting factor FIX is altered; in a fourth human hepatocyte cell, expressing DNA for human clotting factor FX and encoding human clotting factor FX gene product in an engineered, non-naturally occurring Clustered Regularly Interspaced Short Palindromic Repeats (CRISPR)-Synergistic Activation Module (SAM) system, comprising one or more vectors comprising a seventh regulatory element operable in the fourth human hepatocyte cell operably linked to a nucleotide sequence encoding a CRISPR-Cas system guide RNA that hybridizes with human clotting factor FX DNA sequence; an eighth regulatory element operable in the fourth human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA, whereby the guide RNA targets and hybridizes to the human clotting factor FX sequence, whereby expression of gene product for human clotting factor FX is altered; and, wherein none of the Cas9 proteins and the guide RNAs naturally occur together; and combining the gene products of a)-d) in a composition. This is referred to herein as the “singleplex” approach, system or method.

In the methods described herein, the RNA stem aptamer loop can be the Lambda Nut-L aptamer, although any suitable RNA stem aptamer loop that targets and hybridizes as described herein can be used.

In an embodiment, a method of producing a recombinant human clotting factor FII can comprise: expressing DNA for human clotting factor FII and encoding the respective gene product in an engineered, non-naturally occurring Clustered Regularly Interspaced Short Palindromic Repeats (CRISPR)-Synergistic Activation Module (SAM) system in a human hepatocyte cell, comprising a vector comprising a first regulatory element operable in the human hepatocyte cell operably linked to a nucleotide sequence encoding a CRISPR-Cas system guide RNA that hybridizes with human clotting factor FII DNA sequence; and a second regulatory element operable in the human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, and aptamers capable to form RNA aptamer stem loops into the respective guide RNAs and which specifically bind/hybridize to human clotting factor FII, whereby the guide RNA targets and hybridizes to human clotting factor FII sequence, whereby expression of gene products for human clotting factor FII is increased.

In an embodiment, a method of producing a recombinant human clotting factor FVII can comprise: expressing DNA for human clotting factor FVII and encoding the respective gene product in an engineered, non-naturally occurring Clustered Regularly Interspaced Short Palindromic Repeats (CRISPR)-Synergistic Activation Module (SAM) system in a human hepatocyte cell, comprising a vector comprising a first regulatory element operable in the human hepatocyte cell operably linked to a nucleotide sequence encoding a CRISPR-Cas system guide RNA that hybridizes with human clotting factor FVII DNA sequence; and a second regulatory element operable in the human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, and aptamers capable to form RNA aptamer stem loops into the respective guide RNAs and which specifically bind/hybridize to human clotting factor FVII, whereby the guide RNA targets and hybridizes to human clotting factor FVII sequence, whereby expression of gene products for human clotting factor FVII is increased.

In an embodiment, a method of producing a recombinant human clotting factor FIX can comprise: expressing DNA for human clotting factor FIX and encoding the respective gene product in an engineered, non-naturally occurring Clustered Regularly Interspaced Short Palindromic Repeats (CRISPR)-Synergistic Activation Module (SAM) system in a human hepatocyte cell, comprising a vector comprising a first regulatory element operable in the human hepatocyte cell operably linked to a nucleotide sequence encoding a CRISPR-Cas system guide RNA that hybridizes with human clotting factor FIX DNA sequence; and a second regulatory element operable in the human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, and aptamers capable to form RNA aptamer stem loops into the respective guide RNAs and which specifically bind/hybridize to human clotting factor FIX, whereby the guide RNA targets and hybridizes to human clotting factor FIX sequence, whereby expression of gene products for human clotting factor FIX is increased.

In an embodiment, a method of producing a recombinant human clotting factor FX can comprise: expressing DNA for human clotting factor FX and encoding the respective gene product in an engineered, non-naturally occurring Clustered Regularly Interspaced Short Palindromic Repeats (CRISPR)-Synergistic Activation Module (SAM) system in a human hepatocyte cell, comprising a vector comprising a first regulatory element operable in the human hepatocyte cell operably linked to a nucleotide sequence encoding a CRISPR-Cas system guide RNA that hybridizes with human clotting factor FX DNA sequence; and a second regulatory element operable in the human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, and aptamers capable to form RNA aptamer stem loops into the respective guide RNAs and which specifically bind/hybridize to human clotting factor FIX, whereby the guide RNA targets and hybridizes to human clotting factor FX sequence, whereby expression of gene products for human clotting factor FX is increased.

In an embodiment, the methods can further comprise harvesting the human clotting factor produced, and optionally, combining into a composition.

In an embodiment, the Cas9 protein and the guide RNAs do not naturally occur together.

In an embodiment, the CRISPR-Cas system guide RNA that hybridizes with human clotting factor FII DNA sequence comprises the nucleic acid sequence of any one of SEQ ID NOs: 8-16.

In an embodiment, the CRISPR-Cas system guide RNA that hybridizes with human clotting factor FVII DNA sequence comprises the nucleic acid sequence of any one of SEQ ID NOs: 17-21.

In an embodiment, the CRISPR-Cas system guide RNA that hybridizes with human clotting factor FIX DNA sequence comprises the nucleic acid sequence of any one of SEQ ID NOs: 22-28.

In an embodiment, the CRISPR-Cas system guide RNA that hybridizes with human clotting factor FX DNA sequence comprises the nucleic acid sequence of any one of SEQ ID NOs: 29-38.

In an embodiment, the aptamer stem loop is a MS2 aptamer. The MS2 aptamer can comprise the nucleic acid sequence of SEQ ID NO: 1.

In an embodiment, the aptamer stem loop is a Lambda Nut-L aptamer. The Lambda Nut-L aptamer can comprise the nucleic acid sequence of SEQ ID NO: 2.

In an embodiment, the aptamer stem loop is a Lambda Nut-R aptamer. The Lambda Nut-R aptamer can comprise the nucleic acid sequence of SEQ ID NO: 3.

In an embodiment, the aptamer stem loop is a Ob aptamer. The Ob aptamer can comprise the nucleic acid sequence of SEQ ID NO: 4.

In an embodiment, the aptamer stem loop is a BIV TAR aptamer. The BIV TAR aptamer can comprise the nucleic acid sequence of SEQ ID NO: 5.

In an embodiment, the aptamer stem loop is a STNV aptamer. The STNV aptamer can comprise the nucleic acid sequence of SEQ ID NO: 6.

In an embodiment, the aptamer stem loop is a PP7 aptamer. The PP7 aptamer can comprise the nucleic acid sequence of SEQ ID NO: 7.

In an embodiment, a method for producing a recombinant human fibrinogen can comprise: in a human cell, expressing DNA for human fibrinogen and encoding human clotting factor fibrinogen gene product in an engineered, non-naturally occurring Clustered Regularly Interspaced Short Palindromic Repeats (CRISPR)-Synergistic Activation Module (SAM) system, comprising one or more vectors comprising a first regulatory element operable in the human cell operably linked to a nucleotide sequence encoding a CRISPR-Cas system guide RNA that hybridizes with fibrinogen DNA sequence domains, wherein the fibrinogen DNA sequence domains comprises alpha, beta, and gamma domains; a second regulatory element operable in the human cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA, wherein the guide RNA targets and hybridizes to the human fibrinogen DNA sequence domains, wherein expression of gene product for human fibrinogen is increased. The human cell can be a HEK293 cell or a hepatocyte cell.

In an embodiment, the CRISPR-Cas system guide RNA that hybridizes with human fibrinogen DNA sequence domain comprises the nucleic acid sequence of any one of SEQ ID NOs: 39-47 and binds the alpha domain.

In an embodiment, the CRISPR-Cas system guide RNA that hybridizes with human fibrinogen DNA sequence domain comprises the nucleic acid sequence of any one of SEQ ID NOs: 48-55 and binds the beta domain.

In an embodiment, the CRISPR-Cas system guide RNA that hybridizes with human fibrinogen DNA sequence domain comprises the nucleic acid sequence of any one of SEQ ID NOs: 56-63 and binds the gamma domain.

In an embodiment, an isolated nucleic acid sequence can comprise any one of the nucleic acid sequences of SEQ ID NO: 1-67. The nucleic acid can comprise the nucleic acid sequence of SEQ ID NO: 1. The nucleic acid can comprise the nucleic acid sequence of SEQ ID NO: 2 or 3. The nucleic acid can comprise the nucleic acid sequence of SEQ ID NO: 4. The nucleic acid can comprise the nucleic acid sequence of SEQ ID NO: 5. The nucleic acid can comprise the nucleic acid sequence of SEQ ID NO: 6. The nucleic acid can comprise the nucleic acid sequence of SEQ ID NO: 7. The nucleic acid can comprise the nucleic acid sequence of any one of SEQ ID NOs: 8-16. The nucleic acid can comprise the nucleic acid sequence of any one of SEQ ID NOs: 17-21. The nucleic acid can comprise the nucleic acid sequence of any one of SEQ ID NOs: 22-28. The nucleic acid can comprise the nucleic acid sequence of any one of SEQ ID NOs: 29-38. The nucleic acid can comprise the nucleic acid sequence of any one of SEQ ID NOs: 39-47. The nucleic acid can comprise the nucleic acid sequence of any one of SEQ ID NOs: 48-55. The nucleic acid can comprise the nucleic acid sequence of any one of SEQ ID NOs: 56-63. The nucleic acid can comprise the nucleic acid sequence of any one of SEQ ID NO: 64. The nucleic acid can comprise the nucleic acid sequence of any one of SEQ ID NO: 65. The nucleic acid can comprise the nucleic acid sequence of any one of SEQ ID NO: 66 or 67.

In an embodiment, an isolated vector can comprise the isolated nucleic acid of any one of claims 33-49.

In an embodiment, an isolated vector can comprise a plurality of nucleic acids sequence comprising the nucleic acid sequences of SEQ ID NO: 1-67.

In an embodiment, an isolated vector can comprise a plurality of nucleic acids sequence comprising the nucleic acid sequences of SEQ ID NO: 8-38. The vector can comprise any one of nucleic acid sequences of SEQ ID NOs: 8-16; any one of nucleic acid sequences of SEQ ID NOs: 17-21; any one of nucleic acid sequences of SEQ ID NOs: 22-28; and any one of nucleic acid sequences of SEQ ID NOs: 29-38.

In an embodiment, an isolated vector can comprise a plurality of nucleic acids sequence comprising the nucleic acid sequences of SEQ ID NO: 39-63. The vector can comprise any one of nucleic acid sequences of SEQ ID NOs: 39-47; any one of nucleic acid sequences of SEQ ID NOs: 48-55; and any one of nucleic acid sequences of SEQ ID NOs: 56-63.

In an embodiment, an isolated host cell comprise the vector described herein.

In the methods described herein, fibrinogen can be included in the end composition, for instance by being produced in the multiplex system (e.g., in human hepatocyte cell, expressing DNA for fibrinogen under control of the same operable promoter or different operable promoter), or in the singleplex system (e.g., in a fifth human hepatocyte cell, expressing DNA for fibrinogen), or added separately into the final composition.

A method for mitigating coagulopathy in a patient can comprise administering a therapeutically effective amount of at least one of the hepastatic compositions described herein. A method for treating coagulopathy in a patient can comprise administering a therapeutically effective amount of at least one of the hepastatic compositions described herein.

A method of controlling or mitigating hemorrhage in a patient can comprise administering a therapeutically effective amount of at least one of the hepastatic compositions described herein. A method for treating a hemorrhage in a patient can comprise administering a therapeutically effective amount of at least one of the hepastatic compositions described herein.

A method of promoting fluid resuscitation in a patient can comprise administering a therapeutically effective amount of at least one of the hepastatic compositions described herein.

A human hepatocyte cell line that sequentially expresses human clotting factors FII, FVII, FIX and FX is described herein.

### BRIEF DESCRIPTION OF THE FIGURES

FIG. 1: Synergistic Activation Mediator (SAM) system validation and optimization by multi-locus gene activation A. Illustration of SAM system components. B. Three plasmid SAM system expression design. C. HEK293 cell transfection approach. D. mRNA-based gene expression analysis by qRT-PCR. Candidate aptamer and cognate peptide pairs were substituted into the MS2 SAM to initiate Ascl1 and MyoD1 gene overexpression in HEK293 cells (n=3 individual experiments).

FIG. 2: Lambda SAM singleplex & multiplex gene overexpression. A. Factor II, VII, IX, and X gRNA design for singleplex activation. B. mRNA-based gene expression analysis by qRT-PCR illustrating factor II, VII, IX and X transcript expression in Lambda SAM treated HEK293 cells compared to untreated WT cells. (n=3 independent experiments) C. Factor II, VII, IX, and X gRNA design for multiplex activation. D. mRNA-based gene expression analysis by qRT-PCR illustrating a simultaneous upregulation of factor II, VII, IX and X transcripts in Lambda SAM treated HEK293 cells compared to untreated WT cells (n=2 independent experiments). p values were calculated using Student's unpaired, two-sided t-test to compare treated and untreated cells (n.s. p>0.05, *p≤0.05, **p≤0.01, ***p≤0.001, ****p≤0.0001).

FIG. 3: Lambda SAM overexpression of coagulation factors IX and X translates into secreted protein expression. ELISA based protein quantification of transiently overexpressed coagulation factors IX and X in HEK293 cells compared to WT cells (n=3 independent experiments. p values were calculated using Student's unpaired, two-sided t-test to compare treated and untreated cells (n.s. p>0.05, *p≤0.05, **p≤0.01, ***p≤0.001, ****p≤0.0001).

FIG. 4: Lambda SAM singleplex & multiplex fibrinogen overexpression. A. Fibrinogen Alpha, Beta, and Gamma chain gRNA design for singleplex activation. B. mRNA-based gene expression analysis by qRT-PCR illustrating substantial increase in Alpha, Beta, and Gamma transcripts in Lambda SAM treated HEK293 cells compared to untreated WT cells. (n=3 independent experiments) C. Fibrinogen Alpha, Beta, and Gamma chain gRNA design for multiplex activation. D. mRNA-based gene expression analysis by qRT-PCR illustrating simultaneous upregulation of Alpha, Beta, and Gamma transcripts in Lambda SAM treated HEK293 cells compared to untreated WT cells (n=2 independent experiments). p values were calculated using Student's unpaired, two-sided t-test to compare treated and untreated cells (n.s. p>0.05, *p≤0.05, **p≤0.01, ***p≤0.001, ****p≤0.0001).

FIG. 5: CRISPR/Cas9 basics. Gene editing, including insertions/deletions (indels), activation, repression, alternative splicing, etc., is achieved via binding of guide RNA (gRNA) sequence at user-defined locations within the genome. The Cas9 endonuclease binds and creates double stranded DNA break. The system relies on repair via nonhomologous end joining (NHEJ) versus homology directed repair (HDR).

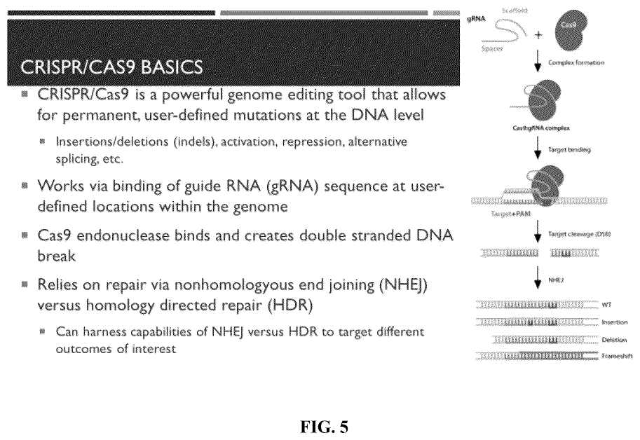

FIG. 6: mechanism of CRISPR/Cas9-SAM. Using hepatocytes, gRNA targets regions upstream of Coagulation Factors 2, 7, 9, 10 and binds. Specifically engineered Cas9 endonuclease designed to bind but not cleave DNA (dCas9) binds DNA/RNA complex. Our dCas9 utilized has been engineered to be part of a synergistic activation module (SAM) which is a fusion complex of dCas9, powerful transcriptional activation domains, RNA stem loop aptamers within the gRNA, and various transcriptional elements. When the SAM is in place, it allows for robust gene activation and allows the cell's native machinery to undergo transcription/translation. Output solution contains highly concentrated coagulation factors that is purified and tested.

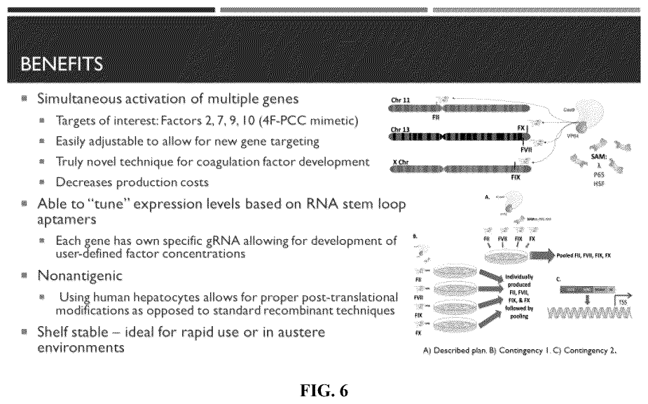

FIG. 7: Benefits of the CRISPR/Cas9-SAM. With this system is simultaneous activation of multiple genes. This includes targets of interest: Factors 2, 7, 9, 10 (4F-PCC mimetic), but is easily adjustable to allow for new gene targeting. It is truly a technique for coagulation factor development, which decreases production costs compared to known methods. This method enables researchers to “tune” expression levels based on RNA stem loop aptamers. Each gene has own specific gRNA allowing for development of user-defined factor concentrations. The product is nonantigenic, because it uses human hepatocytes which allows for proper post-translational modifications as opposed to standard recombinant techniques. The gene products are shelf stable, which is ideal for rapid use or in austere environments (e.g., military field use, emergency crisis, etc.). As the figure also shows, the method works preferably by producing the gene products in a pooled environment (“A) Described plan”), or via producing individual gene products and then pooling/combining (“B) Contingency 1”, or via artificial introduction of gene promotors upstream of the targeted genes using CRISPR associated homology directed repair insertion techniques (“C) Contingency 2”).?

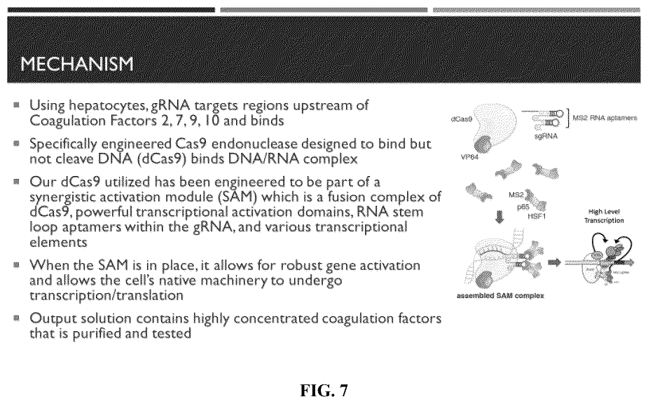

FIG. 8: Preliminary results in 293 FT cell line.

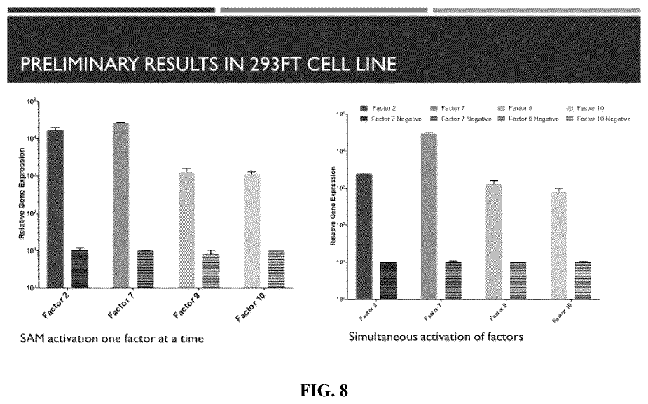

Using the gene activation platform, each targeted factor demonstrated robust overexpression compare to the cells native expression patterns. The graph on the left demonstrates the gene expression upregulation of each factor tested when the activation platform was used as a singleplex entity in each cell (i.e. only targeting one gene per cell). The graph on the right highlights the gene activation platform results demonstrating the robust overexpression of all four factors when tested in a multiplex fashion (i.e targeting all four genes responsible for each protein in each cell).

### DETAILED DESCRIPTION

### Coagulation Peptide Products

The pooled plasma of multiple unrelated donors is the primary source of coagulation peptide products. Hurdles that contribute to the overall challenge of sourcing blood products from this mixed plasma pool include the need for stringent pathogen screening and the comparatively low concentration of peptides both of which contribute to the product's exorbitant cost. Maintaining a stable blood and plasma supply remains a continuous challenge for even the most well-resourced hospitals and blood banks a challenge exacerbated by events such as pandemics and mass casualty that further increase demand. As such, there is a critical need for engineering solutions for the safe, stable, and low-cost production of recombinant coagulation peptides.

Recombinant DNA (rDNA)-based production of hemostatic agents represents an engineering advance that improves supply, safety, and product uniformity without the need for human donors. Recombinant production strategies are based on the introduction of a candidate gene into a cultured cell line for supranormal gene expression and concomitant protein production.

Mammalian and prokaryotic platforms exist; however, the importance of post translational modifications for peptide activity makes mammalian systems attractive. A key posttranslational modification is glycosylation, and this process differs between mammalian species. One of the major cell lines employed for industrial scale production of recombinant human proteins has been the Chinese Hamster Ovary (CHO) cell. Because these cells are not of human origin, the recombinant peptides produced in CHO (or other non-human) cell lines can be immunogenic upon repeat administration. Since treating coagulopathy often requires repeat dosing, this represents a key drawback for non-human cells for the production of recombinant peptides.

Numerous human cell lines have been pursued for use in rDNA strategies and required cellular attributes include ease of culture, robust gene transfer efficiency, and ability to properly produce and modify (e.g., glycosylate, fold, etc.) peptides for therapeutic use and effect. One highly desirable cell type is the human embryonic kidney (HEK293) line that has shown potential as an effective platform capable of producing coagulation factors VIII and IX. By definition recombinant technology employs DNA encoding candidate genes for introduction into cells.

Often these genes are borne on plasmids, small, circular species of DNA, that can be assembled with regulatory (promoter, polyadenylation signal, etc.) and gene expression components.

Plasmids can be isolated at high concentration and purity and upon delivery to the cell, the plasmid regulatory components promote supranormal gene expression of the candidate gene. Because many coagulation therapeutics are multi-component products this individual peptide approach requires multiple cell lines to generate the therapeutic cocktail. This approach is neither cost effective nor efficient and a lack of robust engineering approaches for multicandidate protein production is a persistent hurdle.

Current methods do not address needs for ready and inexpensive access to hemostatic compositions useful for coagulopathy, resuscitation, and general plasma needs. coagulation resuscitation agents are obtained from plasma pooled from unrelated donors making them subject to shortages and necessitating costly pathogen screening. The lack of a stable, affordable, and safe source of coagulation agents represents a therapeutic and engineering gap. Recombinant DNA (rDNA)-based production of proteins represents an engineering solution to improve supply and safety. Currently, rDNA human cell platforms utilize a one gene:one protein approach that is limiting for multi-protein therapeutics. Because most coagulation agents are multi-component this requires multiple corresponding cell lines to generate the therapeutic cocktail.

Recombinant techniques broadly consist of introducing a candidate gene into a cell for overexpression and subsequent protein production. CHO cells represent a widely used cell line for production of recombinant therapeutic proteins and have well-established gene transfer, peptide expression, and purification processes. Mammalian cell systems such as the CHO cell are crucial for producing functional peptides as they enable proper folding and post translational modifications that prokaryotic systems do not provide. A key posttranslational modification is glycosylation, and this process differs between mammalian species. As such, the peptides produced in CHO (or other non-human) cell lines with disparate glycosylation patterns, compared to human cells, can be immunogenic upon repeat administration that is often necessitated in achieving coagulation.

The inventors applied an engineering strategy to the various hurdles in the current technologies, devising a transcriptome modulation approach employing the clustered regularly interspaced short palindromic repeats (CRISPR)/Cas9 platform. CRISPR/Cas9 is a programmable DNA binding system where Cas9 is directed to a user defined genomic target site using a small RNA transcript termed a guide RNA (gRNA). gRNAs contain a ˜15-20-bp sequence which corresponds to the target DNA sequence in the genome and binds via Watson-Crick base pairing. An ˜80 nucleotide scaffold sequence is also present in the gRNA and is bound by Cas9 to form the functional complex. Both the gRNA and Cas9 are permissive to modifications to enable enhanced properties such as genome, epigenome, and transcriptome editing. A powerful transcriptome editing system termed Synergistic Activation Mediators (SAM) has been described that employs a nuclease deficient version of Cas9 while maintaining DNA binding capability.

The catalytically inactive Cas9 (dCas9) from Streptococcus pyogenes fused to the viral particle 64 (VP64) transcriptional activation domain can initiate transcription in a user defined fashion and that activity level can be potentiated with SAM modules. SAMs are comprised of the Heat Shock Factor 1 (HSF1) and Nuclear Factor NF-kappa-B p65 subunit (p65). Both p65 and HSF1 serve to attract transcriptional coactivators to the basal Cas9-VP64 transcription complex and promote high-level target gene expression.

The inventors used cellular engineering using CRISPR Cas9-based SAM for a multiplexed HEK293-based system for overexpression of PCC (factors II, VII, IX, and X) and, in preferred embodiments, fibrinogen. The inventors' strategy represents an approach for producing a user defined recombinant peptide mixture in human cells. The inventors applied it to clotting factors that potentially obviates the need for mixed donor pooled plasma products thus addressing an engineering and therapeutic gap in the field of coagulation factor replacement therapy.

The inventors explored HEK293 cells as a candidate that can, as a human cell, synthesize and modify (e.g., glycosylate) complex human proteins. These cells have become a leading human cell line for laboratory-scale peptide production due to their ease of culture and high plasmid transfection efficiency. An especially useful variant of HEK cells is the 293T cell line which expresses the SV40 large T antigen. Plasmids containing the SV40 origin of replication undergo extrachromosomal amplification within the 293T cells, which can lead to higher levels of transient peptide expression. As such, plasmid-based expression platforms represent an approach for recombinant peptide production in human cells. In these strategies a candidate gene is assembled in a plasmid with elements that enable gene expression (i.e., promoter, polyadenylation signal). While plasmids are easily assembled, the need for a one candidate: one gene engineering approach is cumbersome as it requires a redesigned approach for each new peptide target. The design process includes isolating the desired gene sequence, optimizing the sequence for expression, coupling it with the proper regulatory elements to ensure high level expression, and assembling it into the proper vector for expression in the target cell. Once a plasmid is developed it must be mass produced and isolated with high purity both of which contribute to the overall cost. Plasmids are especially limited for multi-domain genes and multiprotein expression systems where simultaneous expression of multiple genes is desired.

These situations would either require multiple plasmids to be produced for each desired gene or synthesis of a single large plasmid that further complicates the engineering process and may reduce efficiency or cause toxicity during gene delivery.

Toward optimizing protein production in human cells and streamlining the constituent components to coordinate supranormal peptide production, the inventors tested the CRISPR Cas9 SAM system. CRISPR/Cas9 serves as part of a prokaryotic immune defense system used to recognize and degrade infecting bacteriophage by introducing double stranded breaks in specific DNA sequences. The DNA sequence recognition properties of CRISPR/Cas9 have been repurposed for mammalian cell use and the targeting elements are comprised of a ˜20 bp gRNA which is complementary to a sequence in the genome. An ˜80 nucleotide scaffold sequence containing the gRNA forms a complex with the Cas9 endonuclease enzyme at the target sequence where it causes a double stranded break in the DNA. For the purposes of gene activation, the endonuclease properties of Cas9 have been inactivated resulting in a catalytically inactive ‘dead’ Cas9 that retains DNA binding capability. Because Cas9 is permissive to domains being fused to it without a decrement in DNA binding ability, multiple applications for genome, transcriptome, and epigenome engineering exist based on this dCas9 architecture.

One robust application has been the MS2 SAM system that contains a 130 amino acid MS2 affinity peptide that must dimerize before it can be bound by its corresponding aptamer.

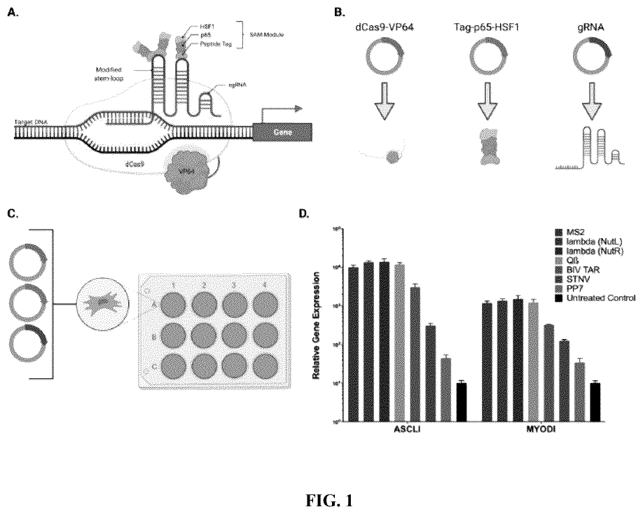

This dimerization causes two copies of the transcriptional activators p65 and HSF1 to be recruited to the SAM complex; and because the association: dissociation rate in this context is unknown, the possibility exists steric or orientation-based effects are operative that could inhibit transcriptional activation activity. Therefore, the inventors investigated six smaller aptamer/affinity peptide monomers to minimize the size and components of the SAM system and constructed the candidates shown in Table 1. Resultant gene expression analysis at two well described MS2 SAM candidate genes (Ascl1 and MyoD1) in HEK293 cells revealed 1.4-fold and 1.3-fold improvement respectively in gene expression mediated by our identified Lambda candidates compared to MS2 (FIG. 1D).

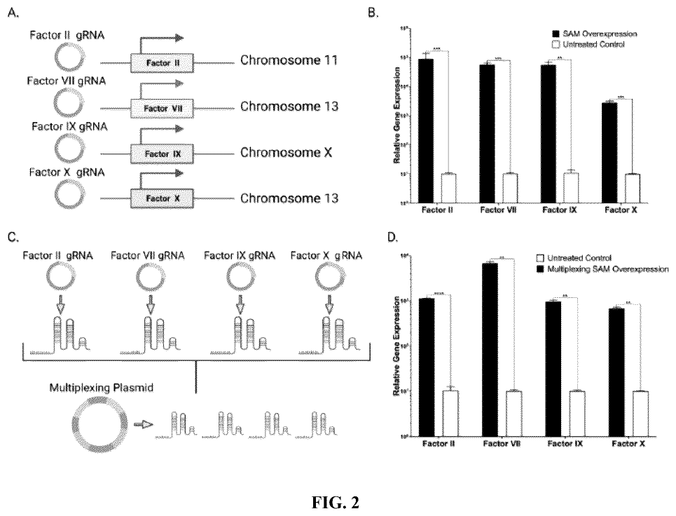

The inventors leveraged these Lambda SAM candidates to achieve singleplex gene activation of coagulation factors II, VII, IX, and X (FIG. 2A,B). These coagulation factor targets were selected as the primary components of prothrombin complex concentrate. The results showed a surprising 1,200-fold increase in factor II, 4,700-fold increase in factor VII, 1,700-fold increase in factor IX, and 120-fold increase in factor X mRNA expression over untreated HEK293 cells. Given the results with singleplex gene activation we then pursued a multiplexing approach that would enable simultaneous upregulation of factors II, VII, IX, and X. To reduce the amount of DNA to be delivery and streamline delivery of six plasmids (dCas9-VP64, Lambda-p65-SF1, four factor guides) the inventors constructed a single multiplexing plasmid to express all four gRNAs for coagulation factors II, VII, IX, and X simultaneously. The results show a surprisingly robust gene activation across all four factor encoding genes compared to untreated controls in HEK293 cells.

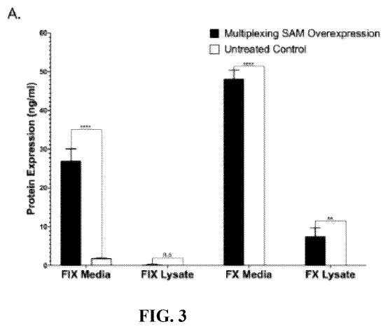

The inventors rank ordered the candidate genes for quantification of peptide production based on a review of the literature and initial methodology screening that shows robust ELISA methodologies for factors IX and X. The inventors observed that our Lambda SAM mediated gene overexpression translates into supraphysiological protein expression (FIG. 3).

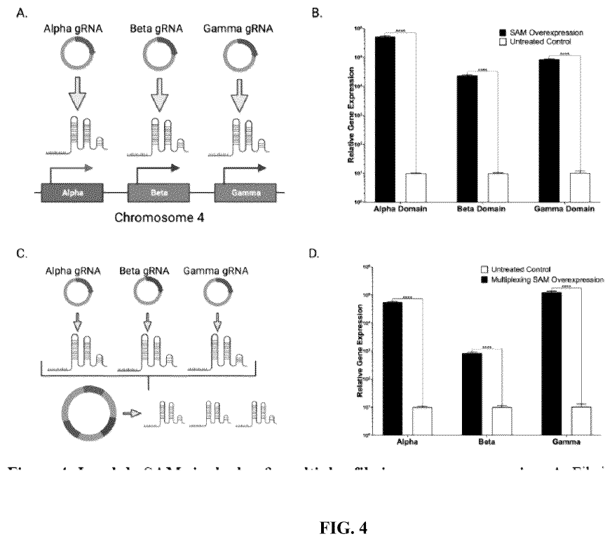

Studies have shown that coadministration of fibrinogen along with PCC may be effective in the management of trauma-related bleeding, reducing the requirements for additional allogenic blood products, and reducing mortality. Therefore, to further validate and extend the system described herein the inventors pursued the use of Lambda SAM to mediate fibrinogen upregulation. Fibrinogen is composed of Alpha, Beta, and Gamma chains which are each encoded by individual sub-units. The results demonstrated robust gene overexpression of each fibrinogen sub-unit when targeted individually (FIG. 4A) or simultaneously (FIG. 4B). These findings show that the CRISPR SAM platform described herein can be used for therapeutic coagulation peptide production.

The CRISPR SAM platform has demonstrated promise as an engineering advance capable of driving targeted gene overexpression with limited engineering component requirements. This makes our system well suited for industrial scale production that often employ large culture volumes and serum free conditions. Importantly, the HEK293 platform is amenable to serum free bioreactor scale culture.

In the experiments, the inventors used CRISPR Cas9 based SAM mediated fibrinogen and factor II, VII, IX, and X prothrombin complex to concentrate singleplex and multiplex supranormal gene expression in a HEK293 cell platform. The approach analyzed multiple aptamer candidates and the inventors identified preferred gRNAs for each gene in support of the production of therapeutic peptide cocktails.

These data represent engineering advances that improve current approaches for recombinant peptide production in human cells and yield a simplified overexpression platform that can be readily adapted to single or multi-protein output with limited engineering requirements. The methods described herein can be used for altering current clinical coagulation products and practices to reduce coagulopathy associated challenges. The modularity of the system as well as identification of aptamer systems that operated across a range of activity levels represents a powerful system for production and tuning of candidate genes that can be broadly applied. In sum, the ability to mediate supranormal expression of single or multiple genes simultaneously represents a facile system for eukaryotic and prokaryotic platforms.

TABLE 1
Aptamer Sequences

| | | Peptide | |
|---|---|---|---|
| Candidate | Aptamer Sequence | Size(aa) | SID |
| MS2 | AACATGAGGATCACCCATGTC | 130 | 1 |
| Lambda | GGGCCCTGAAGAAGGGCCC | 42 | 2 |
| Nut-L | | | |
| Lambda | GCCCTGAAAAAGGGC | 42 | 3 |
| Nut-R | | | |
| Qb | AATGCATGTCTAAGACAGCAT | 133 | 4 |
| BIV TAR | GGCTCGTGTAGCTCATTAGCTCCGAGCC | 17 | 5 |
| STNV | CCTTTTCAAGACATGCAACAATGCACACAG | 196 | 6 |
| PP7 | GGAGCAGACGATATGGCGTCGCTCC | 125 | 7 |

TABLE 2
gRNA Guide Sequences

| Name | Species | Sequence | SID |
|---|---|---|---|
| Factor II Guide 1 | Human | GGACAGGTCCTCCTGGGAGC | 8 |
| Factor II Guide 2 | Human | TCCTGTCCATCTCCACCATC | 9 |
| Factor II Guide 3 | Human | TCTAGGAGGAGTCACAAAAA | 10 |
| Factor II Guide 4 | Human | CGCTATATGTTAGGATTCGA | 11 |
| Factor II Guide 5 | Human | GGCGAGGGGCAACTTCCATC | 12 |
| Factor II Guide 6 | Human | CACACTATGGCGCACGTCCG | 13 |
| Factor II Guide 7 | Human | TATCTACCCACCCGTCCCCA | 14 |
| Factor II Guide 8 | Human | AACATTAACCCAGAGGGGTC | 15 |
| Factor II Guide 9 | Human | ACATGAAGAAATCAGCGGAG | 16 |
| Factor VII Guide 1 | Human | ACCCCAGCTGTGCTGCAGAG | 17 |
| Factor VII Guide 2 | Human | GCTCAGTGGCTGGGCAGCAG | 18 |
| Factor VII Guide 3 | Human | TGCATGGCCACTGGCCGGCC | 19 |
| Factor VII Guide 4 | Human | AACTTTGCCCGTCAGTCCCA | 20 |
| Factor VII Guide 5 | Human | TTGCCAGCGTGCAGGTGTTA | 21 |
| Factor IX Guide 1 | Human | AAATTTCACCTCTGATTTCG | 22 |
| Factor IX Guide 2 | Human | CAGACTCAAATCAGCCACAG | 23 |
| Factor IX Guide 3 | Human | CTTGGGACAAGTGAAGAGAA | 24 |
| Factor IX Guide 4 | Human | CTCTCTGACAAAGATACGGT | 25 |
| Factor IX Guide 5 | Human | TCATCAGGATCTAGTGTTAG | 26 |
| Factor IX Guide 6 | Human | AATAGTCCAAAGACCCATTG | 27 |
| Factor IX Guide 7 | Human | CTTTCACAATCTGCTAGCAA | 28 |
| Factor X Guide 1 | Human | AGGCAGGGGAGTGACCACGC | 29 |
| Factor X Guide 2 | Human | GATGAGGATGAGGTTCTGCG | 30 |
| Factor X Guide 3 | Human | TCTGGCCGCCCAGCTCTGCC | 31 |
| Factor X Guide 4 | Human | GTGGTCTCATGTCACGGGGG | 32 |
| Factor X Guide 5 | Human | AGGTGGTCTCATGTCACGGG | 33 |
| Factor X Guide 6 | Human | AGGCCGCTGGCAGTGAGCGT | 34 |
| Factor X Guide 7 | Human | ACAGTACTCGGCCACACCAT | 35 |
| Factor X Guide 8 | Human | AGCAGGTCTCGGCTCCAATC | 36 |
| Factor X Guide 9 | Human | AGGTCTCGGCTCCAATCAGG | 37 |
| Factor X Guide 10 | Human | CGCTGGGAACACATCCACTC | 38 |
| Fibrinogen Alpha | Human | GGAGGAACAAAGGACGTAGA | 39 |
| Guide 1 | | | |
| Fibrinogen Alpha | Human | TCGGAAGCAAACAAGCTGAG | 40 |
| Guide 2 | | | |
| Fibrinogen Alpha | Human | TTCTGGGATACCAACAGCAT | 41 |
| Guide 3 | | | |
| Fibrinogen Alpha | Human | CAAGAGGTTTGGTTAATCAT | 42 |
| Guide 4 | | | |
| Fibrinogen Alpha | Human | TTTCAGCTGGAGTGCTCCTC | 43 |
| Guide 5 | | | |
| Fibrinogen Alpha | Human | CTTCCGATAAGCTGTTGCAA | 44 |
| Guide 6 | | | |
| Fibrinogen Alpha | Human | GAGATCTCCTAATTGTTGAC | 45 |
| Guide 7 | | | |
| Fibrinogen Alpha | Human | TGATTCTCCAGTCAACAATT | 46 |
| Guide 8 | | | |
| Fibrinogen Alpha | Human | GGAGGGTTGACTGTCTACAC | 47 |
| Guide 9 | | | |
| Fibrinogen Beta | Human | AAGATACACATCTCTCTTTG | 48 |
| Guide 1 | | | |
| Fibrinogen Beta | Human | GTAAATTTGACCTACTCACA | 49 |
| Guide 2 | | | |
| Fibrinogen Beta | Human | TTAGTGGTTGCCTTGTGAGT | 50 |
| Guide 3 | | | |
| Fibrinogen Beta | Human | CTGCTAGGAATGACTTCAGA | 51 |
| Guide 4 | | | |
| Fibrinogen Beta | Human | AGCAAAGCTTATTTACTTGT | 52 |
| Guide 5 | | | |
| Fibrinogen Beta | Human | CAGAGGACTCAGATATATAT | 53 |
| Guide 6 | | | |
| Fibrinogen Beta | Human | TCAGTTAAGTCTACATGAAA | 54 |
| Gudie 7 | | | |
| Fibrinogen Beta | Human | GGACAAAATGATGGGAAGTT | 55 |
| Guide 8 | | | |
| Fibrinogen Gamma | Human | AAGCATTATCCATTGCTGTG | 56 |
| Guide 1 | | | |
| Fibrinogen Gamma | Human | CTCCTTTTTGGCCCAGCTCA | 57 |
| Guide 2 | | | |
| Fibrinogen Gamma | Human | TGATCCAAAATTATCTGCAA | 58 |
| Guide 3 | | | |
| Fibrinogen Gamma | Human | GGCCCCCAACCTATACTGTC | 59 |
| Guide 4 | | | |
| Fibrinogen Gamma | Human | AATGGCTGGAGCTGATCACG | 60 |
| Guide 5 | | | |
| Fibrinogen Gamma | Human | GGGAACCTGACAGTATAGGT | 61 |
| Guide 6 | | | |
| Fibrinogen Gamma | Human | ACGGGGCCTCCTTACCAGAA | 62 |
| Guide 7 | | | |
| Fibrinogen Gamma | Human | CACTACAAGGCTCGGAGCTC | 63 |
| Guide 8 | | | |
| Ascl1 Guide 1 | Human | GCAGCCGCTCGCTGCAGCAG | 64 |
| MyoD1 Guide 1 | Human | GGGCCCCTGCGGCCACCCCG | 65 |
| Factor X Guide 1 | Mouse | AAGGGTAGCCTCCCCTAGAA | 66 |
| Factor X Guide 2 | Mouse | GGCCCAGGGCTCCAATCAGA | 67 |

The method described herein produces a wholly in vitro-derived composition of four recombinant clotting factors represents a stable off the shelf product that is derived from the plasma of multiple human donors. Available standards of care for achieving hemostasis and reversing coagulopathy are accomplished through the use of human derived blood products to directly replace the depleted clotting factors. Whole blood fractionation to obtain these products is costly, time consuming, and non-uniform due to its obtainment from pooled whole blood sources. The demand for plasma continues to increase and, for instance, the requirement to treat one hemophilia patient for one year in non-trauma scenarios is ˜1,000 plasma donations. This high demand makes plasma a strategic resource defined as “an economically important raw material which is subject to a higher risk of supply interruption.” Disruptive events such as mass casualties or the ongoing COVID-19 pandemic can strain the availability of volunteer donors.

A compounding impediment to standardized plasma administration in the current paradigm is that pooled sources require extended processing (i.e., filtration and heating) and lot testing for pathogen removal and detection. These measures mitigate, but do not eliminate the potential for pathogen transmission, and contribute to the significant cost of 4F-PCC therapy. Treating coagulopathy with plasma products also requires thawing and rapid administration such that the storage and delivery of these products limit them as frontline military therapeutics. Additionally, pooling donors can result in transfusion reactions that may be exacerbated in the trauma setting where repetitive transfusions are often mandated. The compositions described herein are improved because they are not derived from whole blood, and are entirely recombinant. The compositions and its components are not naturally occurring. The compositions described herein have the advantages of being easily stored, have a lengthy shelf life, are field deployable, and are produced in a uniform manner in human cells that would allow for production without risk of disruption or serial administration without adverse immunological consequences.

Known 4F-PCC products derived from whole blood donated from various persons, are comprised of the clotting factors II, VII, IX, and X and have been shown to reverses elevated international normalized ratios (INR) in trauma patients with warfarin-induced coagulopathy and that the effects can last ≥48 hours. With the reported shelf life of ˜36 months, 4F-PCC holds great promise as a field deployable hemostatic agent. However, the current practice of obtaining 4F-PCC from pooled donor human plasma remains a hurdle for secure, cost effective application for worldwide use.

The recombinant production of clotting factors represents an engineering advance but most clotting factors are produced individually and in non-human cells. Recombinant DNA (rDNA)-based production represents a more standardized engineering approach and is based on introducing a candidate gene into a cell line for protein production. rDNA approaches can lead to supranormal gene and protein expression levels. Examples such as the production of Factor VIII and Factor IX for hemophilia A and B, respectively, have shown potential. Chinese hamster ovary (CHO) cells are one of the most commonly utilized cell lines for rDNA therapeutic peptide production. However, therapeutic peptides produced in non-human cell lines may contain post-translational modifications such as glycan epitopes that are immunogenic when used in humans. In particular, the Gala1,3-gal and N-glycolylneuraminic acid residues expressed by CHO-produced recombinant clotting factors have demonstrated antigenicity with most people showing seropositivity.

Conversely, the use of human cells for rDNA techniques results in the appropriate non-antigenic, post-translational modifications. However, only single coagulation factors are currently produced by these recombinant measures requiring the generation of multiple cell lines, each of which are costly and require labor intensive batch testing for clinical scaling and manufacturing. The use of non-human cells to produce single recombinant peptides on a candidate-to-candidate basis represents an engineering and therapeutic gap. There is an urgent need to create a standardized human cell line that produces multiple clotting factors simultaneously in order to support hemostatic interventions for military personnel.

With a goal to obviate the need for human blood component creation, the inventors developed a method to produce emostatic compositions comprised of recombinant human clotting factors. The inventors' work resulted in pharmacologic agents and compositions for hemorrhage control, coagulopathy reversal, resuscitation, and plasma alternative. The hemostatic agents and compositions are not derived from whole blood, and have the advantages of being shelf stable, cost-efficient, antigen-free, and useful as a universally accepted plasma alternative.

The inventors used oCRISPR/Cas9 techniques in a cellular engineering approach to drive expression of multiple genes simultaneously. This required development of a system, the CRISPR/Cas9-based Synergistic Activation Mediator (SAM), for a multiplexed human cell-based system for overexpression of coagulation factors II, VII, IX, X. Preferably, fibrinogen is also expressed and a component of the final product.

CRISPR/Cas9 is a powerful genome editing tool that allows for permanent, user-defined mutations at the DNA level-such as insertions/deletions (indels), activation, repression, alternative splicing, etc. The CRISPR/Cas9 tool works via binding of guide RNA (gRNA) sequence at user-defined locations within the genome. The Cas9 endonuclease binds and creates double stranded DNA break. CRISPR relies on repair via nonhomologous end joining (NHEJ) versus homology directed repair (HDR). The inventors sought to harness certain capabilities of NHEJ versus HDR to target different outcomes of interest.

In efforts to resolve these engineering and therapeutic gaps, this methods described herein utilize hepatocytes as an engineering platform for CRISPR/Cas9 engineering. The CRISPR/Cas9 system represents a bacterial adaptive immune system that is mobilized in response to phage infection which has been repurposed for mammalian cell use. CRISPR/Cas9 functions as an RNA-guided DNA binding complex comprised of a small guide RNA (gRNA) transcript that complexes with the Cas9 endonuclease protein to target a specific protospacer adjacent motif (PAM).

By inactivating the nuclease components of Cas9 and fusing four copies of the VP16 transcriptional activation domain to create a VP64 tetramer complex, endogenous/candidate gene transcription can be upregulated at user directed sites. Gene activation/transcription can be further amplified using a unique synergistic activation module (SAM). A SAM incorporates RNA aptamer stem loops into the gRNA to specifically bind defined peptide motifs with high fidelity. As described here, the SAM construct uses unique DNA aptamers, and we were able to improve the honing mechanisms for the transcriptional activation process. By combining the use of a SAM and the multiplex capabilities of the CRISPR/Cas9 system, overexpression of numerous genes at the genome scale can be achieved. This allows for specified, user-defined gene upregulation and protein expression. The methods described herein are drawn to the application of CRISPR/Cas9 for the simultaneous production of therapeutic clotting factors. The inventors designed a multigene activation for therapeutic peptide production by choosing specific reagents in hepatocytes to engineer a uniform coagulation factor-based resuscitation solution resulting from the upregulation of multiple hepatocyte-derived coagulation factors. Engineered human cells that produce supranormal levels of multiple clotting factors and possess the requisite posttranslational modifications resulted in a highly desirable product with significant translational impact to trauma and critical care resuscitation practices.

The approach used by the inventors is in contrast to other recombinant techniques that employ lentiviral gene transfer or plasmid DNA containing the candidate gene. Lentiviral gene transfer requires production of viral particles and laborious clonogenic isolation of cells for assessing gene/peptide expression. Moreover, viral vectors can be subject to gene silencing that may negatively impact their ability to produce optimal levels of the gene. Thus, the inventors circumvented the hurdles associated with the current cellular engineering techniques.

Ultimately, the resuscitation fluid can be useful to mitigate the need for blood product utilization while providing a shelf stable product to help improve resuscitation, reverse coagulopathy, and promote hemostasis in a fiscally responsible and safe way.

In the approach developed by the inventors, components of the CRISPR-SAM system were identified, optimized, and expressed in Human Embryonic Kidney Cells (HEK293). Singleplex and multiplex gene expression was assessed by qRT-PCR and subsequent protein expression by ELISA. The results were unexpectantly good. Singleplex activation yielded 120-4,700-fold increase in gene expression across factors II, VII, IX, and X while multiplex assays resulted in a 60-680-fold increase. When fibrinogen was a component, the fibrinogen subunit activation resulted in a 1,700-92,000-fold and 80-5,500-fold increase in singleplex and multiplex assays, respectively. Factors IX and X increased from 0-7 ng/ml to 27 ng/mL and 48 ng/mL in control and treated HEK293 cells, respectively. This demonstrated CRISPR/Cas9-based SAM mediated factor II, VII, IX, X, and fibrinogen singleplex and multiplex supranormal gene expression in a human cell platform. This system and methods offers improved recombinant protein production techniques in human cells and is a simplified overexpression platform readily adapted to single or multi-protein output. The SAM system adds to the armamentarium of CRISPR/Cas9 engineering tools that can be brought to bear to produce therapeutic mixtures.

The invention provides for a hemostatic composition comprising recombinant non-whole blood derived, human clotting factors FII, FVII, FIX and FX. The clotting factors are preferably free of other proteins, free of antigens that would be present in filtered plasma, are non-antigenic for humans, and have only human post-translational modifications (e.g, contain only the post-translational modifications consistent with primary human proteins that are derived in vivo). The compositions described herein comprise a combination of clotting factors, in that it is free of other proteins that would be present in compositions where the clotting factors are derived from whole blood and is free of non-human post-translational modifications that would be present in clotting factors produced in non-human cell lines (e.g., standard recombinant processes which use bacterial cells or yeast as production systems). Another advantage of the compositions described herein is that they have a high and titratable concentration of each factor, along with varied ratios of each factor as desired. Preferably, the hemostatic compositions also includes fibrinogen, either added independently/separately to the composition or produced in the unique method along with the four clotting factors. The inclusion of fibrinogen is an additional component and advantage not found in the current PCC formulations. Preferably, the hemostatic composition is shelf-stable at room temperature for up to 36 months.

The hemostatic composition described herein can be used as a human plasma alternative.

### EXAMPLES

### Materials and Methods

### SAM Vector Assembly:

Plasmids carrying dCas9-VP64 (46912) and MS2-p65-HSF1 (61426) were purchased from Addgene (Watertown, Massachusetts). The gRNA scaffold plasmid was synthesized based on previous efforts6. The genes of interest were isolated and integrated into the mammalian expression vector pcDNA3.1 by Gibson Assembly using HiFi DNA Assembly Master Mix from New England BioLabs (Ipswich, Massachusetts) per the manufacturer's instructions. MS2, Qb, PP7, Lambda, STNV, and BIV TAR stem loop and aptamer DNA sequences were synthesized by IDT (Coralville, Iowa) and assembled into a gRNA backbone in a pcDNA3.1 expression in a similar fashion.

### Cell Culture & Transfection Reagents:

HEK293 cells, grown in DMEM complete media, were obtained from ATCC (CRL-1573) (Manassas, Virginia). The adherent cells were grown as a monolayer in T75 cm2 Corning (Corning, New York) cell culture treated flasks and detached with Trypsin-EDTA solution (0.25%) obtained from Invitrogen (Waltham, Massachusetts). Transient transfections utilized Lipofectamine 2000 from Invitrogen seeding 100,000 cells per well in a Corning 24-well plate.

### Quantitative RT-PCR:

Total mRNA was isolated using the RNeasy Plus kit from Qiagen (Hilden, Germany). Equal amounts of RNA were reverse transcribed Superscript VILO-IV from Invitrogen (Waltham, Massachusetts). Gene expression analysis was carried out on a QuantStudio 3 realtime

PCR system from Applied Biosystems (Waltham, Massachusetts). Taqman gene expression master mix and probes were used from Applied Biosystems.

### Protein Quantification:

Conditioned media and cell lysate were collected from treated HEK293 cells. Cells were lysed in Reporter Lysis Buffer from Promega (Madison, Wisconsin) for 30 minutes before shearing through a 27.5-gauge needle. Both cell lysate and media were cleared of cellular debris by centrifugation before analysis. ELISA kits factor IX (ab108831) and factor X (ab108832) were purchased from Abcam (Cambridge, United Kingdom). Analysis was conducted on a Tecan Infinite Microplate reader (Männedorf, Switzerland) using Magellan software.

### Graphing, Statistical Analysis, and Images:

All graphs were constructed using GraphPad Prism software v.8. Statistical analyses were performed using unpaired two-sided t-tests to compare the treated and untreated cells in transfection experiments. Images were constructed and formatted using BioRender.

### Example 1

### Clustered Regularly Interspaced Short Palindromic Repeats/Cas9-Synergistic Activation Module

Fused to nuclease inactive ‘dead’ Cas9 (dCas9) are four repeats of the VP16 herpes simplex viral protein VP16 that enable potent gene activation in tandem with SAMs comprised of p65 and HSF1 (FIG. 1A) 5. While dCas9 directs the complex to a user directed (e.g., promoter) region, aptamer sequences incorporated into the gRNA can recruit and bind the cognate peptide fused to p65 and HSF1. These aptamer sequences and their affinity peptides are commonly found in viruses and the first described SAMs were derived from the bacteriophage MS2. The inventors constructed and tested six additional naturally occurring aptamers: Bacteriophage Qb (QB), Pseudomonas Phage PP7 (PP7), Enterobacteria Phage Lambda (Lambda Nut-L and Nut-R), Satellite Tobacco Necrosis Virus (STNV), and Bovine Immunodeficiency Virus (BIV TAR) 7-11 (Table 1).

The inventors designed a gRNA plasmid with candidate aptamer stem loops and a plasmid carrying the corresponding affinity peptide fused to p65 & HSF1 (FIG. 1B). Plasmids carrying SAM, gRNA, and dCas9-VP64 (FIG. 1C) were introduced into HEK293 cells and assessed for their ability to upregulate two candidate genes (Ascl1 and MyoD1) which were previously used to validate the MS2 SAM (FIG. 1D) 5. The inventors observed variability in the SAMs' ability to overexpress genes Ascl1 and MyoD1 in the human genome as assessed by quantitative RTPCR (qRT-PCR). Of the candidates, the Lambda Nut-R aptamer/peptide, which unlike the MS2 peptide does not require dimerization to be recognized by its aptamer, showed a 1.4-fold increase for Ascl1 and a 1.3-fold increase for MyoD1, respectively compared to MS2 (FIG. 1D). These data demonstrate that Lambda Nut-R mediated the highest gene expression levels and was therefore pursued as the lead candidate for the remainder of the study.

### Example 2

### Lambda Sam Shows Potential for Prothrombin Complex Concentrate Production

Having optimized the components of the SAM system the inventors next sought to define the positional effects of candidate gRNAs to mediate multigene activation of the coagulation factors II, VII, IX, and X that are key constituents of 4 factor-PCC (4F-PCC). The inventors designed nine factor II, five factor VII, seven factor IX, and nine factor X candidate gRNAs, positioned within ˜600 bp of the initiating methionine transcriptional start site (FIG. 2A). Each respective gene gRNA plasmid was constructed with the Lambda aptamer sequences incorporated into the stem loops of the gRNA and transiently delivered into HEK293 cells along with Cas9-VP64, p65, and HSF1 (FIG. 1C). Seventy-two hours after gene delivery, RNA was analyzed by qRT-PCR for quantification of the target transcript. Shown in FIG. 2B is the data from the observed best gRNA that mediated overexpression ranging from 100-4,700-fold increase across the four coagulation factors compared to the untreated HEK293 controls.

With optimized gRNAs identified and validated in a singleplex gene overexpression assay, the inventors next combined all the gene specific gRNA plasmids and evaluated their ability to promote gene overexpression in a tandem/multiplex fashion by delivering them to the same population of cells. To accomplish this, the inventors constructed a multiplex gRNA plasmid expressing the optimized gRNAs for factor II, VII, IX, and X (FIG. 2C). The gRNA concatemer plasmid was simultaneously delivered with dCas9-VP64 and Lambda-p65-HSF1. Under these conditions, the inventors observed between 60-680-fold increase in expression across the four factors compared to untreated control in HEK293 cells. These results show that the Lambda SAM system can facilitate simultaneous high level multiplexed gene expression of the factors II, VII, IX, and X genes in HEK293 cells (FIG. 2D).

### Example 3

### Lambda SAM Drives Factor IX and X Protein Overexpression

Next, the inventors undertook efforts to test whether the overexpression observed at the mRNA level translated into recombinant human protein. The inventors transiently expressed the Lambda SAM using the optimized gRNAs for factor IX and X and analyzed the resulting supernatant and cellular lysate via ELISA. The targeted factors were predominantly prevalent as secreted products with 27+/−7 ng/ml of factor IX and 48+/−5 ng/ml of factor X secreted into the media across three biological replicates (FIG. 3). These data demonstrate that SAM-mediated gene overexpression produced the corresponding peptide at supraphysiological levels.

### Example 4

### Lambda SAM Drives Activation of Multi-Domain Gene Fibrinogen

Showing the potential of the SAM system for upregulating genes for the 4F-PCC components, the inventors sought to extend the application by determining the ability of SAMs to upregulate other relevant coagulation proteins. Fibrinogen, an essential coagulation factor in the formation of stable clots, is commonly used in tandem with PCC to achieve homeostatic coagulation. It was hypothesized that multi-domain genes such as Fibrinogen, composed of Alpha, Beta, and Gamma domains, would benefit from our multiplex SAM approach. The inventors designed and evaluated nine Alpha, eight Beta, and eight Gamma gRNAs that were delivered with our optimized Lambda SAM system into HEK293 cells. A surprisingly 1,700-92,000-fold increase in expression was observed across the three domain encoding genes with the best candidates (FIG. 4B). Similar to the results for factors II, VII, IX, and X it showed that overexpression of sub-unit genes such as fibrinogen can be achieved by targeting each of the domains individually. Preferred gRNAs were identified in this singleplex gene activation to multiplex targeting using a ‘all in one’ gRNA plasmid (FIG. 4C). An unexpected 80-5,500-fold increase in expression across the three domains as compared to the untreated control in HEK293 cells was observed. These findings show that multi-domain genes such as fibrinogen can also be multiplexed to drive simultaneous high level gene overexpression (FIG. 4D).

Although the invention has been described in some detail by way of illustration and example for purposes of clarity of understanding, it should be understood that certain changes and modifications can be practiced within the scope of the appended claims. Modifications of the above-described modes for carrying out the invention that would be understood in view of the foregoing disclosure or made apparent with routine practice or implementation of the invention to persons of skill in molecular biology and related fields are intended to be within the scope of the following claims.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

While the foregoing invention has been described in connection with this preferred aspect, it is not to be limited thereby but is to be limited solely by the scope of the claims which follow.

1. A method of producing a recombinant human clotting factors FII, FVII, FIX and FX, comprising the step of:
expressing DNA for each of human clotting factors FII, FVII, FIX and FX and encoding the respective gene products in an engineered, non-naturally occurring Clustered Regularly Interspaced Short Palindromic Repeats (CRISPR)-Synergistic Activation Module (SAM) system in a human hepatocyte cell, comprising one or more vectors comprising
(a) a first regulatory element operable in the human hepatocyte cell operably linked to a nucleotide sequence encoding a CRISPR-Cas system guide RNA that hybridizes with human clotting factor FII DNA sequence;
a nucleotide sequence encoding a CRISPR-Cas system guide RNA that hybridizes with human clotting factor FVII DNA sequence;
a nucleotide sequence encoding a CRISPR-Cas system guide RNA that hybridizes with human clotting factor FIX DNA sequence; and
a nucleotide sequence encoding a CRISPR-Cas system guide RNA that hybridizes with human clotting factor FX DNA sequence; and
(b) a second regulatory element operable in the human hepatocyte cell operably linked to a nucleotide sequence encoding a synergistic activation module (SAM), which comprises
a nucleotide sequence encoding catalytically inactive Type-II Cas9 protein engineered to bind but not cleave DNA a DNA/RNA complex, and
transcriptional activation domains, and aptamers capable to form RNA aptamer stem loops into the respective guide RNAs and which specifically bind/hybridize to the respective human clotting factor,
whereby each guide RNA targets and hybridizes to the appropriate human clotting factor FII, FVII, FIX or FX sequence, whereby expression of gene products for human clotting factor FII, FVII, FIX and FX is altered; and, wherein the Cas9 protein and the guide RNAs do not naturally occur together.

2. A method for producing a recombinant human clotting factors FII, FVII, FIX and FX, comprising the steps of:
(a) in a first human hepatocyte cell, expressing DNA for human clotting factor FII and encoding human clotting factor FII gene product in an engineered, non-naturally occurring Clustered Regularly Interspaced Short Palindromic Repeats (CRISPR)-Synergistic Activation Module (SAM) system, comprising one or more vectors comprising
a first regulatory element operable in the first human hepatocyte cell operably linked to a nucleotide sequence encoding a CRISPR-Cas system guide RNA that hybridizes with human clotting factor FII DNA sequence;
a second regulatory element operable in the first human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA,
whereby the guide RNA targets and hybridizes to the human clotting factor FIX sequence, whereby expression of gene product for human clotting factor FIX is altered;
(b) in a second human hepatocyte cell, expressing DNA for human clotting factor FVII and encoding human clotting factor FVII gene product in an engineered, non-naturally occurring Clustered Regularly Interspaced Short Palindromic Repeats (CRISPR)-Synergistic Activation Module (SAM) system, comprising one or more vectors comprising
a third regulatory element operable in the second human hepatocyte cell operably linked to a nucleotide sequence encoding a CRISPR-Cas system guide RNA that hybridizes with human clotting factor FVII DNA sequence;
a fourth regulatory element operable in the second human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA,
whereby the guide RNA targets and hybridizes to the human clotting factor FVII sequence, whereby expression of gene product for human clotting factor FVII is altered;
(c) in a third human hepatocyte cell, expressing DNA for human clotting factor FIX and encoding human clotting factor FIX gene product in an engineered, non-naturally occurring Clustered Regularly Interspaced Short Palindromic Repeats (CRISPR)-Synergistic Activation Module (SAM) system, comprising one or more vectors comprising
a fifth regulatory element operable in the third human hepatocyte cell operably linked to a nucleotide sequence encoding a CRISPR-Cas system guide RNA that hybridizes with human clotting factor FIX DNA sequence;
a sixth regulatory element operable in the third human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA,
whereby the guide RNA targets and hybridizes to the human clotting factor FIX sequence,
whereby expression of gene product for human clotting factor FIX is altered;
(d) in a fourth human hepatocyte cell, expressing DNA for human clotting factor FX and encoding human clotting factor FX gene product in an engineered, non-naturally occurring Clustered Regularly Interspaced Short Palindromic Repeats (CRISPR)-Synergistic Activation Module (SAM) system, comprising one or more vectors comprising
a seventh regulatory element operable in the fourth human hepatocyte cell operably linked to a nucleotide sequence encoding a CRISPR-Cas system guide RNA that hybridizes with human clotting factor FX DNA sequence;
an eighth regulatory element operable in the fourth human hepatocyte cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA,
whereby the guide RNA targets and hybridizes to the human clotting factor FX sequence,
whereby expression of gene product for human clotting factor FX is altered; and, wherein none of the Cas9 proteins and the guide RNAs naturally occur together; and
(e) combining the gene products of a)-d) in a composition.

3. A method of producing a recombinant human clotting factor comprising the step of:
expressing DNA for a human clotting factor selected from FII, FVII, FIX, FX, or a combination thereof and encoding the respective gene products in an engineered, non-naturally occurring Clustered Regularly Interspaced Short Palindromic Repeats (CRISPR)-Synergistic Activation Module (SAM) system in a human hepatocyte cell, comprising one or more vectors comprising
(a) a first regulatory element operable in the human hepatocyte cell operably linked to
a nucleotide sequence encoding a CRISPR-Cas system guide RNA that hybridizes with human clotting factor FII DNA sequence;
a nucleotide sequence encoding a CRISPR-Cas system guide RNA that hybridizes with human clotting factor FVII DNA sequence;
a nucleotide sequence encoding a CRISPR-Cas system guide RNA that hybridizes with human clotting factor FIX DNA sequence;
a nucleotide sequence encoding a CRISPR-Cas system guide RNA that hybridizes with human clotting factor FX DNA sequence; or
a combination thereof;
(b) a second regulatory element operable in the human hepatocyte cell operably linked to
a nucleotide sequence encoding a synergistic activation module (SAM), which comprises
a nucleotide sequence encoding catalytically inactive Type-II Cas9 protein engineered to bind but not cleave DNA a DNA/RNA complex, and
transcriptional activation domains, and aptamers capable to form RNA aptamer stem loops into the respective guide RNAs and which specifically bind/hybridize to the respective human clotting factor,
whereby each guide RNA targets and hybridizes to the appropriate human clotting factor FII, FVII, FIX, FX sequence, or a combination thereof,
whereby expression of gene products for human clotting factor FII, FVII, FIX, FX, or a combination thereof is increased.

4. The method of any one of claims 1-3, wherein the method further comprises harvesting the human clotting factor produced, and optionally, combining into a composition.

5. The method of any one of claims 1-4, wherein the Cas9 protein and the guide RNAs do not naturally occur together.

6. The method of any one of claims 1-5, wherein the CRISPR-Cas system guide RNA that hybridizes with human clotting factor FII DNA sequence comprises the nucleic acid sequence of any one of SEQ ID NOs: 8-16.

7. The method of any one of claims 1-5, wherein the CRISPR-Cas system guide RNA that hybridizes with human clotting factor FVII DNA sequence comprises the nucleic acid sequence of any one of SEQ ID NOs: 17-21.

8. The method of any one of claims 1-5, wherein the CRISPR-Cas system guide RNA that hybridizes with human clotting factor FIX DNA sequence comprises the nucleic acid sequence of any one of SEQ ID NOs: 22-28.

9. The method of any one of claims 1-5, wherein the CRISPR-Cas system guide RNA that hybridizes with human clotting factor FX DNA sequence comprises the nucleic acid sequence of any one of SEQ ID NOs: 29-38.

10. The method of any one of claims 1-9, wherein the aptamer stem loop is a MS2 aptamer.

11. The method of claim 10, wherein the MS2 aptamer comprises the nucleic acid sequence of SEQ ID NO: 1.

12. The method of any one of claims 1-9, wherein the aptamer stem loop is a Lambda Nut-L aptamer.

13. The method of claim 12, wherein the Lambda Nut-L aptamer comprises the nucleic acid sequence of SEQ ID NO: 2.

14. The method of any one of claims 1-9, wherein the aptamer stem loop is a Lambda Nut-R aptamer.

15. The method of claim 14, wherein the Lambda Nut-R aptamer comprises the nucleic acid sequence of SEQ ID NO: 3.

16. The method of any one of claims 1-9, wherein the aptamer stem loop is a Ob aptamer.

17. The method of claim 16, wherein the Ob aptamer comprises the nucleic acid sequence of SEQ ID NO: 4.

18. The method of any one of claims 1-9, wherein the aptamer stem loop is a BIV TAR aptamer.

19. The method of claim 18, wherein the BIV TAR aptamer comprises the nucleic acid sequence of SEQ ID NO: 5.

20. The method of any one of claims 1-9, wherein the aptamer stem loop is a STNV aptamer.

21. The method of claim 20, wherein the STNV aptamer comprises the nucleic acid sequence of SEQ ID NO: 6.

22. The method of any one of claims 1-9, wherein the aptamer stem loop is a PP7 aptamer.

23. The method of claim 22, wherein the PP7 aptamer comprises the nucleic acid sequence of SEQ ID NO: 7.

24. A hemostatic composition comprising recombinant human clotting factors FII, FVII, FIX and FX made by the method of any one of claims 1-23.

25. A human plasma alternative comprising the hemostatic composition of claim 24.

26. A method for producing a recombinant human fibrinogen comprising the steps of:
in a human cell, expressing DNA for human fibrinogen and encoding human clotting factor fibrinogen gene product in an engineered, non-naturally occurring Clustered Regularly Interspaced Short Palindromic Repeats (CRISPR)-Synergistic Activation Module (SAM) system, comprising one or more vectors comprising
(a) a first regulatory element operable in the human cell operably linked to a nucleotide sequence encoding a CRISPR-Cas system guide RNA that hybridizes with fibrinogen DNA sequence domains,
wherein the fibrinogen DNA sequence domains comprises alpha, beta, and gamma domains;
(b) a second regulatory element operable in the human cell operably linked to a nucleotide sequence encoding a synergistic activation module (SAM), which comprises a nucleotide sequence encoding catalytically inactive Type-II Cas9 protein engineered to bind but not cleave DNA a DNA/RNA complex, and transcriptional activation domains, optionally comprising transcriptional activation subunits, and an aptamer capable to form an RNA aptamer stem loop into the guide RNA,
wherein the guide RNA targets and hybridizes to the human fibrinogen DNA sequence domains,
wherein expression of gene product for human fibrinogen is increased.

27. The method of claim 26, wherein the human cell is a HEK293 cell or a hepatocyte cell.

28. The method of claim 26 or 27, wherein the CRISPR-Cas system guide RNA that hybridizes with human fibrinogen DNA sequence domain comprises the nucleic acid sequence of any one of SEQ ID NOs: 39-47 and binds the alpha domain.

29. The method of any one of claims 26-28, wherein the CRISPR-Cas system guide RNA that hybridizes with human fibrinogen DNA sequence domain comprises the nucleic acid sequence of any one of SEQ ID NOs: 48-55 and binds the beta domain.

30. The method of any one of claims 26-29, wherein the CRISPR-Cas system guide RNA that hybridizes with human fibrinogen DNA sequence domain comprises the nucleic acid sequence of any one of SEQ ID NOs: 56-63 and binds the gamma domain.

31. A hemostatic composition comprising recombinant fibrinogen made by the method of any one of claims 26-30.

32. A human plasma alternative comprising the hemostatic composition of claim 31.

33. An isolated nucleic acid sequence comprising any one of the nucleic acid sequences of SEQ ID NO: 1-67.

34. The isolated nucleic acid of claim 33, wherein the nucleic acid comprises the nucleic acid sequence of SEQ ID NO: 1.

35. The isolated nucleic acid of claim 33, wherein the nucleic acid comprises the nucleic acid sequence of SEQ ID NO: 2 or 3.

36. The isolated nucleic acid of claim 33, wherein the nucleic acid comprises the nucleic acid sequence of SEQ ID NO: 4.

37. The isolated nucleic acid of claim 33, wherein the nucleic acid comprises the nucleic acid sequence of SEQ ID NO: 5.

38. The isolated nucleic acid of claim 33, wherein the nucleic acid comprises the nucleic acid sequence of SEQ ID NO: 6.

39. The isolated nucleic acid of claim 33, wherein the nucleic acid comprises the nucleic acid sequence of SEQ ID NO: 7.

40. The isolated nucleic acid of claim 33, wherein the nucleic acid comprises the nucleic acid sequence of any one of SEQ ID NOs: 8-16.

41. The isolated nucleic acid of claim 33, wherein the nucleic acid comprises the nucleic acid sequence of any one of SEQ ID NOs: 17-21.

42. The isolated nucleic acid of claim 33, wherein the nucleic acid comprises the nucleic acid sequence of any one of SEQ ID NOs: 22-28.

43. The isolated nucleic acid of claim 33, wherein the nucleic acid comprises the nucleic acid sequence of any one of SEQ ID NOs: 29-38.

44. The isolated nucleic acid of claim 33, wherein the nucleic acid comprises the nucleic acid sequence of any one of SEQ ID NOs: 39-47.

45. The isolated nucleic acid of claim 33, wherein the nucleic acid comprises the nucleic acid sequence of any one of SEQ ID NOs: 48-55.

46. The isolated nucleic acid of claim 33, wherein the nucleic acid comprises the nucleic acid sequence of any one of SEQ ID NOs: 56-63.

47. The isolated nucleic acid of claim 33, wherein the nucleic acid comprises the nucleic acid sequence of any one of SEQ ID NO: 64.

48. The isolated nucleic acid of claim 33, wherein the nucleic acid comprises the nucleic acid sequence of any one of SEQ ID NO: 65.

49. The isolated nucleic acid of claim 33, wherein the nucleic acid comprises the nucleic acid sequence of any one of SEQ ID NO: 66 or 67.

50. An isolated vector comprising the isolated nucleic acid of any one of claims 33-49.

51. An isolated vector comprising a plurality of nucleic acids sequence comprising the nucleic acid sequences of SEQ ID NO: 1-67.

52. An isolated vector comprising a plurality of nucleic acids sequence comprising the nucleic acid sequences of SEQ ID NO: 8-38.

53. The vector of claim 52, wherein the vector comprises
any one of nucleic acid sequences of SEQ ID NOs: 8-16;
any one of nucleic acid sequences of SEQ ID NOs: 17-21;
any one of nucleic acid sequences of SEQ ID NOs: 22-28; and
any one of nucleic acid sequences of SEQ ID NOs: 29-38.

54. An isolated vector comprising a plurality of nucleic acids sequence comprising the nucleic acid sequences of SEQ ID NO: 39-63.

55. The vector of claim 54, wherein the vector comprises
any one of nucleic acid sequences of SEQ ID NOs: 39-47;
any one of nucleic acid sequences of SEQ ID NOs: 48-55; and
any one of nucleic acid sequences of SEQ ID NOs: 56-63.

56. An isolated host cell comprising the vector of any one of claims 50-55.

57. A method for mitigating coagulopathy in a patient comprising administering a therapeutically effective amount of the hepastatic composition of claim 24.

58. A method for mitigating coagulopathy in a patient comprising administering a therapeutically effective amount of the hepastatic composition of claim 31.

59. A method for treating coagulopathy in a patient comprising administering a therapeutically effective amount of the hepastatic composition of claim 24.

60. A method for treating coagulopathy in a patient comprising administering a therapeutically effective amount of the hepastatic composition of claim 31.

61. A method for controlling or mitigating hemorrhage in a patient comprising administering a therapeutically effective amount of the hepastatic composition of claim 24.

62. A method for controlling or mitigating hemorrhage in a patient comprising administering a therapeutically effective amount of the hepastatic composition of claim 31.

63. A method for treating a hemorrhage in a patient comprising administering a therapeutically effective amount of the hepastatic composition of claim 24.

64. A method for treating a hemorrhage in a patient comprising administering a therapeutically effective amount of the hepastatic composition of claim 31.

65. A method for promoting fluid resuscitation in a patient comprising administering a therapeutically effective amount of the hepastatic composition of claim 24.

66. A method for promoting fluid resuscitation in a patient comprising administering a therapeutically effective amount of the hepastatic composition of claim 31.
